(19) United States
(12) Patent Application Publication
(10) Pub. No.: US 2024/0008517 A1
(43) Pub. Date: Jan. 11, 2024

(54) COMPOSITIONS AND METHODS FOR PRESERVING PROBIOTIC VIABILITY

(71) Applicant: MJN U.S. HOLDINGS LLC, Evansville, IN (US)

(72) Inventors: Maciej Witalis CHICHLOWSKI, Evansville, IN (US); Gabriele GROSS, Nijmegen (NL)

(73) Assignee: MJN U.S. HOLDINGS LLC, Evansville, IN (US)

(21) Appl. No.: 17/780,358

(22) PCT Filed: Dec. 4, 2020
(86) PCT No.: PCT/EP2020/084759

§ 371 Date: May 26, 2022

(30) Foreign Application Priority Data
Dec. 4, 2019 (GB) 1917735.1

(51) Int. Cl. A23L 33/135; A23L 33/00; C12N 1/04; A23L 33/115; C12N 1/20

(52) U.S. Cl. CPC A23L 33/135; A23L 33/40; C12N 1/04; A23L 33/115; C12N 1/20; C12R 2001/225

(57) ABSTRACT

The present application provides compositions and methods comprising milk fat globule membrane (MFGM) for use in preserving probiotic viability and/or improving probiotic tolerance to bile stress.

### FIELD OF THE INVENTION

The present application relates to compositions and methods for preserving probiotic viability. In particular, the present application relates to compositions and methods for preserving probiotic viability in the presence of bile.

### BACKGROUND

Probiotics are microorganisms, such as bacteria or yeast, which have been shown to exert a beneficial effect on the health of a host subject, in certain instances. As a result, probiotics are typically included in a wide range of products, including paediatric nutrition products and supplements. However, there are many challenges associated with the use of probiotics in paediatric nutrition and other nutritional products. An example of one such challenge is maintenance of probiotic viability in the mammalian gastrointestinal (GI) system e.g. the human GI system.

In some cases, viability of the probiotic microorganism is required to exert beneficial health effects, for example where the metabolic activity of the probiotic is involved in the underlying mechanism of action. In these instances, it can be important to maintain probiotics in a viable state as they travel through the GI system. The viability of probiotics in the GI system is affected by a range of factors including pH, post-acidification during products fermentation, exposure to bile, and exposure to hydrogen peroxide.

The bile present in the GI system can lead to a reduction in the viability of probiotics. Some probiotics go into a ‘stress state’ in response to the presence of bile, in an attempt to prevent degradation by the bile. The probiotic ‘stress state’ can involve an increase in the production of exopolysaccharide (EPS) by the probiotics, to form a capsular polysaccharide (CPS). However, despite this ‘stress response’ there is still usually a significant reduction in probiotic viability in the presence of bile. Further, the presence of the CPS may affect the probiotics' ability to exert a beneficial effect on the health of a host subject. Thus, for probiotics whose ability to exert a meaningful beneficial effect on the host subject is dependent on them being in a viable state, a level of resistance to the bile present in the GI system may be required.

In such cases, an increased survival of probiotics in the GI system could lead to an enhanced beneficial health effect. Compositions and methods that improve the viability of probiotics would therefore be beneficial.

### SUMMARY OF INVENTION

In a first aspect, a composition for use in preserving viability of a probiotic, wherein the composition comprises at least milk fat globule membrane (MFGM), is provided.

Preferably, preserving viability of a probiotic comprises preserving, or limiting the reduction in, viability of the probiotic in the presence of bile.

Preferably, the composition further comprises a probiotic.

Preferably, the probiotic comprises a Bifidobacterium species, a Lactobacillus species, or a combination thereof. More preferably, the probiotic comprises Bifidobacterium longum subsp. infantis, Lactobacillus rhamnosus GG (ATCC number 53103), or a combination thereof. In a preferred aspect, the probiotic comprises Lactobacillus rhamnosus GG (ATCC number 53103).

Preferably, the milk fat globule membrane is provided by an enriched milk product, buttermilk, or a combination thereof. More preferably, the enriched milk product is an enriched whey protein concentrate.

Preferably, the milk fat globule membrane is derived from at least one of bovine milk, bovine cream, porcine milk, equine milk, buffalo milk, goat milk, murine milk, camel milk, or any combination thereof.

Preferably, the composition further comprises a prebiotic. In a preferred aspect, the prebiotic comprises polydextrose, galactooligosaccharides, or a combination thereof.

Preferably, the composition further comprises lactoferrin.

Preferably, the composition further comprises a long-chain polyunsaturated fatty acid. More preferably, the long-chain polyunsaturated fatty acid comprises docosahexaenoic acid, arachidonic acid, or a combination thereof. In a preferred aspect, the long-chain polyunsaturated fatty acid comprises docosahexaenoic acid.

Preferably, the composition further comprises partially hydrolysed protein.

Preferably, the composition further comprises extensively hydrolysed casein.

Preferably, the composition is a synthetic composition.

Preferably, the composition is intended for an adult.

Preferably, the composition is intended for a paediatric subject. The paediatric subject may be an infant. The infant may be an infant delivered by caesarean section (hereinafter referred to as ‘a C-section infant’. Alternatively, the infant may have been vaginally delivered. The infant may be a preterm infant. Alternatively, the infant may be a full-term infant. Suitably, the composition may be an infant formula or the composition may be a follow-up formula. Alternatively, the paediatric subject may be a child. Suitably, the composition may be a follow-up formula or the composition may be a young child milk.

In a second aspect, the use of milk fat globule membrane for preserving viability of a probiotic is provided.

Preferably, preserving viability of a probiotic comprises preserving, or limiting the reduction in, viability of a probiotic in the presence of bile.

Preferably, the probiotic comprises a Bifidobacterium species, a Lactobacillus species, or a combination thereof. More preferably, the probiotic comprises Bifidobacterium longum subsp. infantis, Lactobacillus rhamnosus GG (ATCC number 53103), or a combination thereof. In a preferred aspect, the probiotic comprises Lactobacillus rhamnosus GG (ATCC number 53103).

In a third aspect, the use of milk fat globule membrane for reducing bile-induced stress of a probiotic is provided.

Preferably, the probiotic comprises a Bifidobacterium species, a Lactobacillus species, or a combination thereof. More preferably, the probiotic comprises Bifidobacterium longum subsp. infantis, Lactobacillus rhamnosus GG (ATCC number 53103), or a combination thereof. In a preferred aspect, the probiotic comprises Lactobacillus rhamnosus GG (ATCC number 53103).

In a fourth aspect, a method for preserving viability of a probiotic comprising providing a composition comprising a probiotic and milk fat globule membrane is provided.

Preferably, preserving viability of a probiotic comprises preserving, or limiting the reduction in, viability of the probiotic in the presence of bile.

Preferably, the probiotic comprises a Bifidobacterium species, a Lactobacillus species, or a combination thereof. More preferably, the probiotic comprises Bifidobacterium longum subsp. infantis, Lactobacillus rhamnosus GG (ATCC number 53103), or a combination thereof. In a preferred aspect, the probiotic comprises Lactobacillus rhamnosus GG (ATCC number 53103).

Preferably, the composition further comprises a prebiotic comprising polydextrose and galactooligosaccharides.

Preferably, the composition further comprises lactoferrin.

Preferably, the composition further comprises a long-chain polyunsaturated fatty acid comprising docosahexaenoic acid.

Preferably, the composition is a synthetic composition.

Preferably, the composition is intended for an adult.

Preferably, the composition is intended for a paediatric subject. The paediatric subject may be an infant. The infant may be an infant delivered by caesarean section (hereinafter referred to as ‘a C-section infant’. Alternatively, the infant may have been vaginally delivered. The infant may be a preterm infant. Alternatively, the infant may be a full-term infant. Suitably, the composition may be an infant formula or the composition may be a follow-up formula. Alternatively, the paediatric subject may be a child. Suitably, the composition may be a follow-up formula or the composition may be a young child milk.

In a fifth aspect, a probiotic-stabilised composition, wherein the probiotic-stabilised composition comprises milk fat globule membrane (MFGM) and a probiotic, is provided.

### Definitions

Probiotics can usually be classified as ‘viable’ or ‘non-viable’. The term ‘viable probiotics’ refers to living microorganisms. Probiotics that have been heat-killed, or otherwise inactivated, are termed ‘non-viable probiotics’ i.e. non-living microorganisms.

“Probiotic viability” is a term used to describe whether a microorganism, which has been shown to exert a beneficial effect on the health of a host subject, is in a ‘live’, a ‘dormant/inactivated’, or a ‘dead’ state. “Probiotic viability” is quantified in terms of the number of colony forming units (CFUs) present when a probiotic is cultured. Methods of probiotic culturing are well-known in the art. The method of probiotic culturing may therefore be any method in the art.

The expressions “preserving viability of a probiotic” and “limiting the reduction in viability of a probiotic”, in terms of the present disclosure, mean maintaining the number of probiotic cells in a ‘live state’ at the same, or close to the same, levels in the presence of a disadvantageous external factor (such as the presence of acid, the presence of bile, increased temperature, etc.), as the number of probiotic cells in a ‘live state’ in the absence of said disadvantageous external factor. The present invention is particularly concerned with preserving viability of a probiotic and/or limiting the reduction in viability of a probiotic in the presence of bile i.e. maintaining the number of CFUs, in the presence of bile, at the same level, or close to the same level, as the number of CFUs in the absence of bile.

“Bile-induced stress of a probiotic” means the initiation of a ‘stress state’ of a probiotic, in response to the presence of bile. If a probiotic is bile-sensitive, it may initiate a ‘stress state’ in an attempt to avoid being affected by the bile. In its ‘stress state’, a probiotic can produce exopolysaccharide (EPS), which forms a capsular polysaccharide (CPS) around the probiotic. The “bile-induced stress of a probiotic” can therefore be quantified by determining the amount of EPS produced by the probiotic. Methods of EPS production determination are well-known in the art. The method of EPS production determination may therefore be any method in the art. The “bile-induced stress of a probiotic” can also be quantified by determining the amount of colony forming units (CFUs) present when a probiotic is cultured. The method of probiotic culturing may therefore be any method in the art.

The expression “reducing bile-induced stress ofa probiotic”, in terms of the present disclosure, means reducing the number of probiotic cells that enter a ‘stress state’, reducing the level of ‘stress state’ that the probiotic cells attain, or both, in the presence of bile. The present invention is particularly concerned with reducing the amount of EPS produced by a probiotic in the presence of bile, compared to a previously determined level of EPS production, by a probiotic in the presence of bile. As mentioned previously, the present invention is also concerned with preserving viability of a probiotic and/or limiting the reduction in viability of a probiotic in the presence of bile.

“Milk” means a substance that has been drawn or extracted from the mammary gland of a mammal.

“Milk-based composition” means a composition comprising any milk-derived or milk-based product known in the art. For example, a “milk-based composition” may comprise bovine casein, bovine whey, bovine lactose, or any combination thereof.

“Enriched milk product” generally refers to a milk ingredient that has been enriched with MFGM and/or certain MFGM components, such as proteins and lipids found in the MFGM.

“Nutritional composition” means a substance or composition that satisfies at least a portion of a subject's nutrient requirements. “Nutritional composition(s)” may refer to liquids, powders, gels, pastes, solids, concentrates, suspensions, or ready-to-use forms of enteral formulas, oral formulas, formulas for infants, formulas for paediatric subjects, formulas for children, young child milks, and/or formulas for adults.

The term “synthetic” when applied to a composition, nutritional composition, or mixture means a composition, nutritional composition, or mixture obtained by biological and/or chemical means, which can be chemically identical to the mixture naturally occurring in mammalian milks. A composition, nutritional composition, or mixture is said to be “synthetic” if at least one of its components is obtained by biological (e.g. enzymatic) and/or chemical means.

“Paediatric subject” means a human under 18 years of age. The term “adult”, in terms of the present disclosure, refers to a human that is 18 years of age or greater. The term “paediatric subject” may refer to preterm infants, full-term infants, and/or children, as described below. A paediatric subject may be a human subject that is between birth and 8 years old. In another aspect, “paediatric subject” refers to a human subject between 1 and 6 years of age. Alternatively, “paediatric subject” refers to a human subject between 6 and 12 years of age.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes full-term infants, preterm infants, low birth weight infants, very low birth weight infants, and extremely low birth weight infants. “Preterm” means an infant born before the end of the 37^{th} week of gestation. “Full-term” means an infant born after the end of the 37^{th} week of gestation.

“Child” means a subject ranging in age from 12 months to 13 years. A child may be a subject between the ages of 1 and 12 years old. In another aspect, the terms “children” or “child” may refer to subjects that are between one and about six years old. Alternatively, the terms “children” or “child” may refer to subjects that are between about seven and about 12 years old. The term “young child” means a subject ranging from 1 year to 3 years of age.

“Infant formula” means a composition that satisfies at least a portion of the nutrient requirements of an infant.

“Follow-up formula” means a composition that satisfies at least a portion of the nutrient requirements of an infant from the 6^{th} month onwards, and for young children from 1 to 3 years of age.

“Young child milk”, in terms of the present disclosure, means a fortified milk-based beverage intended for children over one year of age (typically from one to six years of age). Young child milks are designed with the intent to serve as a complement to a diverse diet, to provide additional insurance that a child achieves continual, daily intake of all essential vitamins and minerals, macronutrients plus additional functional dietary components, such as non-essential nutrients that have purported health-promoting properties.

The term “enteral” means deliverable through or within the gastrointestinal, or digestive, tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject's body.

The term “degree of hydrolysis” refers to the extent to which peptide bonds are broken by a hydrolysis method. The degree of protein hydrolysis for the purposes of characterising the hydrolysed protein component of the composition is easily determined by one of ordinary skill in the formulation arts, by quantifying the amino nitrogen to total nitrogen ratio (AN/TN) of the protein component of the selected composition. The amino nitrogen component is quantified by USP titration methods for determining amino nitrogen content, with the total nitrogen component being determined by the Tecator Kjeldahl method. These methods are well-known to one of ordinary skill in the analytical chemistry art.

The term “partially hydrolysed” means having a degree of hydrolysis which is greater than 0% but less than about 50%.

The term “extensively hydrolysed” means having a degree of hydrolysis which is greater than or equal to about 50%.

The term “substantially free” means containing less than a functional amount of the specified component, typically less than 0.1% by weight, and includes 0% by weight of the specified ingredient.

As applied to nutrients, the term “essential” refers to any nutrient that cannot be synthesised by the body in amounts sufficient for normal growth, so it must be supplied by the diet. The term “conditionally essential” as applied to nutrients means that the nutrient must be supplied by the diet when adequate amounts of the precursor compound is unavailable to the body for endogenous synthesis to occur.

The term “viable”, refers to live microorganisms. The term “non-viable” or “non-viable probiotic” refers to non-living probiotic microorganisms, their cellular components and/or metabolites thereof. Such a non-viable probiotic may have been heat-killed or otherwise inactivated, but may still retain the ability to favourably influence the health of the host.

The amount of viable probiotic is detailed in CFUs, with the amount of non-viable probiotic disclosed as probiotic cell equivalents, wherein the term “probiotic cell equivalents” refers to the level of non-viable, non-replicating probiotics equivalent to an equal number of viable cells. The term “non-replicating” is to be understood as the amount of non-replicating microorganisms obtained from the same amount of replicating bacteria (CFU/g), including inactivated probiotics, fragments of DNA, cell wall, cytoplasmic compounds, etc. In other words, the quantity of non-living, non-replicating organisms is expressed in terms of CFU as if all the microorganisms were alive, regardless whether they are dead, non-replicating, inactivated, fragmented, etc. The probiotic source incorporated into the composition may comprise both viable CFUs and non-viable cell-equivalents.

The term “prebiotic” refers to a non-digestible food ingredient that beneficially affects the host by selectively stimulating the growth and/or activity of one or a limited number of bacteria in the digestive tract, which can improve the health of the host. Prebiotics exert health benefits, which may include, but are not limited to: selective stimulation of the growth and/or activity of one or a limited number of beneficial gut bacteria; stimulation of the growth and/or activity of ingested probiotic microorganisms; selective reduction in gut pathogens; and, favourable influence on gut short chain fatty acid profile. The prebiotic of the composition may be naturally-occurring, synthetic, or developed through the genetic manipulation of organisms and/or plants, whether such new source is now known or developed later.

The term “sialic acid” refers to a family of derivatives of neuraminic acid. N-acetylneuraminic acid (Neu5Ac) and N-glycolylneuraminic acid (Neu5Gc) are among the most abundant, naturally-found forms of sialic acid, especially Neu5Ac in human and cow's milk.

The term “organism” refers to any contiguous living system, such as an animal, plant, fungus, or micro-organism.

“Non-human lactoferrin” refers to lactoferrin that is produced by or obtained from a source other than human breast milk.

All percentages, parts, and ratios as used herein are detailed by weight of the total composition, unless otherwise specified. All amounts specified as administered “per day” may be delivered in a single unit dose, in a single serving, or in two or more doses or servings administered over the course of a 24 hour period.

All references to singular characteristics or limitations in the present disclosure shall include the corresponding plural characteristic or limitation, and vice versa, unless otherwise specified or clearly implied to the contrary, by the context in which the reference is made.

All combinations of method or process steps disclosed herein can be performed in any order, unless otherwise specified or clearly implied to the contrary, by the context in which the referenced combination is made.

The compositions and methods of the present disclosure can comprise, consist of, or consist essentially of any of the components described herein, as well as including any additional component useful in nutritional compositions.

### DETAILED DESCRIPTION

MFGM is a naturally occurring bioactive membrane structure that surrounds the fat droplets in human breast milk and other mammalian milk e.g. cow's milk. MFGM is comprised of a trilayer lipid structure that comprises a complex mixture of phospholipids, proteins, glycoproteins, triglycerides, polar lipids, cholesterol, enzymes, and other components.

The inventors have surprisingly found that the number of probiotic colony forming units, in the presence of bile, is much higher when MFGM is present, compared with the absence of MFGM.

The presence of MFGM was found to preserve, or at least limit the reduction in, the viability of a probiotic, in the presence of bile. The viability of a probiotic may be quantified by any of the probiotic culturing methods disclosed in Studies 3, 4, 6, or 7, or any other probiotic culturing method known in the art.

In addition, the presence of MFGM was found to reduce the bile-induced stress of a probiotic. The bile-induced stress of a probiotic may be quantified by the EPS production determination method disclosed in Study 5a, or any other EPS production determination method known in the art.

The discovery that MFGM is able to preserve probiotic viability was not previously known or suggested, and was unexpected.

The composition of the present invention comprises MFGM. The MFGM present in the composition may be provided by an enriched milk product. The enriched milk product may be formed by fractionation of non-human (e.g. bovine) milk. Enriched milk products may have a total protein level of between 20% and 90%; preferably, enriched milk products may have a total protein level of between 68% and 80%. MFGM proteins may account for between 3% and 50% of the enriched milk product protein content; preferably, MFGM proteins may account for 7% to 13% of the enriched milk product protein content.

The enriched milk product may comprise an enriched whey protein concentrate (eWPC). Alternatively, the enriched milk product may comprise an enriched lipid fraction derived from milk. The eWPC and the enriched lipid fraction may be produced by any number of fractionation techniques. These techniques comprise, but are not limited, to melting point fractionation, organic solvent fractionation, super critical fluid fractionation, and any variants and/or any combination thereof. Alternatively, eWPC is available commercially, including under the trade name Lacprodan MFGM-10, available from Arla Food Ingredients of Viby, Denmark. With the addition of eWPC, the lipid composition of infant formulas and other paediatric compositions can more closely resemble that of human milk.

The composition may comprise eWPC at a level of about 0.5 grams per litre (g/L) to about 10 g/L. Preferably, the eWPC is present at a level of about 1 g/L to about 9 g/L. More preferably, eWPC is present in the composition at a level of about 3 g/L to about 8 g/L. Alternatively, the composition may comprise eWPC at a level of about 0.06 grams per 100 kilocalories (g/100 kcal) to about 1.5 g/100 kcal. Preferably, the eWPC is present at a level of about 0.3 g/100 kcal to about 1.4 g/100 kcal. More preferably, the eWPC is present in the composition at a level of about 0.4 g/100 kcal to about 1 g/100 kcal.

The MFGM present in the composition may also be provided by buttermilk. Buttermilk, in the context of the present disclosure, refers to an aqueous by-product of different milk fat manufacturing processes, especially the butter making process. Buttermilk is a concentrated source of MFGM components compared to other milk sources. Buttermilk includes dry buttermilk, which is defined as having a protein content of not less than 30%, and dry buttermilk product, which is defined as having a protein content of less than 30%. Both types of dry buttermilk have a minimum fat content of 4.5% and a moisture maximum of 5%. Cultured buttermilk is also within the contemplation of this disclosure. Buttermilk contains components such as lactose, minerals, oligosaccharides, immunoglobulins, milk lipids, and milk proteins, each of which is found in the aqueous phase during certain dairy cream processing steps.

Buttermilk may be obtained through different processes, such as: churning of cream during production of butter or cheese; production of variants of butter such as sweet cream butter, clarified butter, butterfat; production of anhydrous milk fat (butter oil) from cream or butter; removal of the fat-free dry matter and water from milk, cream, or butter, which is required to make anhydrous milk fat, yields buttermilk as a by-product; removal can be accomplished by mechanical- and/or chemical-induced separation; or, production of anhydrous milk fat (butter oil) from blending secondary skim and β-serum (and/or butter serum) streams together, respectively.

The enriched milk product, buttermilk, or both may be derived from non-human milk sources, such as bovine whole milk, bovine cream, porcine milk, equine milk, buffalo milk, goat milk, murine milk, or camel milk. The composition may be solely a milk-based composition i.e. a non-synthetic composition. Alternatively, the composition may be a synthetic composition.

The composition may comprise one or more probiotics. The probiotic may comprise any Bifidobacterium species, any Lactobacillus species, or a combination thereof. Preferably, the probiotic comprises Bifidobacterium adolescentis (ATCC number 15703), Bifidobacterium animalis subsp. lactis, Bifidobacterium breve, Bifidobacterium longum subsp. infantis (B. infantis), Lactobacillus acidophilus, Lactobacillus gasseri (ATCC number 33323), Lactobacillus reuteri (DSM number 17938), Lactobacillus rhamnosus GG (LGG; ATCC number 53103), or any combination thereof. More preferably, the probiotic comprises LGG or B. infantis, or a combination thereof. Most preferably, the probiotic comprises LGG.

The probiotic may be viable. The amount of probiotic in the composition may vary from about 1×10⁴ CFU to about 1.5×10¹² CFU of probiotic(s) per 100 kcal. Preferably, the amount of probiotic may be from about 1×10⁶ CFU to about 1×10⁹ CFU of probiotic(s) per 100 kcal. More preferably, the amount of probiotic may vary from about 1×10⁷ CFU to about 1×10⁸ CFU of probiotic(s) per 100 kcal.

The composition may comprise one or more prebiotics. The one or more prebiotics may comprise oligosaccharides, polysaccharides, or any other prebiotics that comprise fructose, xylose, soya, galactose, glucose, mannose, or any combination thereof. More specifically, the prebiotic may comprise polydextrose (PDX), polydextrose powder, lactulose, lactosucrose, raffinose, glucooligosaccharides, inulin, fructooligosaccharides, isomaltooligosaccharides, soybean oligosaccharides, lactosucrose, xylooligosaccharides, chitooligosaccharides, mannooligosaccharides, aribino-oligosaccharides, sialyloligosaccharides, fucooligosaccharides, galactooligosaccharides (GOS), and gentiooligosaccharides.

The total amount of prebiotic present in the composition may be from about 1.0 g/L to about 10.0 g/L of the composition. Preferably, the total amount of prebiotic present in the composition may be from about 2.0 g/L and about 8.0 g/L of the composition. Alternatively, the total amount of prebiotic present in the composition may be from about 0.01 g/100 kcal to about 1.5 g/100 kcal. Preferably, the total amount of prebiotic present in the composition may be from about 0.15 g/100 kcal to about 1.5 g/100 kcal.

Preferably, the composition may comprise a prebiotic comprising PDX, GOS, or a combination thereof.

The amount of PDX in the composition may be within the range of from about 1.0 g/L and 10.0 g/L. Preferably, the composition may contain an amount of PDX that is between about 2.0 g/L and 8.0 g/L. The amount of PDX in the composition may be within the range of from about 0.015 g/100 kcal to about 1.5 g/100 kcal. Preferably, the amount of PDX may be within the range of from about 0.2 g/100 kcal to about 0.6 g/100 kcal. Alternatively, the amount of PDX in the composition may be from about 0.05 g/100 kcal to about 1.5 g/100 kcal.

The amount of GOS in the composition may be from about 0.015 g/100 kcal to about 1.0 g/100 kcal. The amount of GOS in the composition may be from about 0.2 g/100 kcal to about 0.5 g/100 kcal.

Preferably, the composition comprises PDX in combination with GOS. Advantageously, the combination of PDX and GOS may stimulate and/or enhance endogenous butyrate production by microbiota. The composition may comprise GOS and PDX in a total amount of at least about 0.015 g/100 kcal. The composition may comprise GOS and PDX in a total amount of about 0.015 g/100 kcal to about 1.5 g/100 kcal. Preferably, the composition may comprise GOS and PDX in a total amount of from about 0.1 g/100 kcal to about 1.0 g/100 kcal. The prebiotic may comprise at least 20% weight per weight (w/w) PDX, GOS, or a combination thereof.

The composition may comprise lactoferrin. The lactoferrin may comprise human lactoferrin produced by a genetically modified organism, non-human lactoferrin, or a combination thereof. The non-human lactoferrin may comprise bovine lactoferrin (bLF), porcine lactoferrin, equine lactoferrin, buffalo lactoferrin, goat lactoferrin, murine lactoferrin, or camel lactoferrin.

Lactoferrin may be present in the composition in an amount of at least about 15 mg/100 kcal. The composition may comprise between about 15 and about 300 mg lactoferrin per 100 kcal. Preferably, the composition may comprise lactoferrin in an amount of from about 60 mg to about 150 mg/100 kcal. More preferably, the composition may comprise about 60 mg to about 100 mg lactoferrin per 100 kcal.

The composition may comprise lactoferrin in an amount of about 0.5 mg to about 1.5 mg per millilitre of formula. Preferably, lactoferrin may be present in quantities of from about 0.6 mg to about 1.3 mg per millilitre of formula. Alternatively, the composition may comprise between about 0.1 g and about 2 g lactoferrin per litre. Preferably, the composition comprises between about 0.6 g and about 1.5 g lactoferrin per litre of formula.

The composition may comprise a source of long-chain polyunsaturated fatty acids (LCPUFAs). The source of LCPUFAs may comprise docosahexaenoic acid (DHA), α-linoleic acid, γ-linoleic acid, linoleic acid, linolenic acid, eicosapentaenoic acid (EPA), arachidonic acid (ARA), or any combination thereof. Preferably, the composition comprises a source of LCPUFAs comprising DHA, ARA, or a combination thereof.

The amount of LCPUFA in the composition may be at least about 5 mg/100 kcal. The composition may comprise LCPUFA in amount from about 5 mg/100 kcal to about 100 mg/100 kcal. Preferably, the composition may comprise LCPUFA in amount from about 10 mg/100 kcal to about 50 mg/100 kcal.

The composition may comprise about 5 mg/100 kcal to about 80 mg/100 kcal of DHA. Preferably, the composition may comprise about 10 mg/100 kcal to about 20 mg/100 kcal of DHA. More preferably, the composition may comprise about 15 mg/100 kcal to about 20 mg/100 kcal of DHA.

The composition may comprise about 10 mg/100 kcal to about 100 mg/100 kcal of ARA. Preferably, the composition may comprise about 15 mg/100 kcal to about 70 mg/100 kcal of ARA. More preferably, the composition may comprise about 20 mg/100 kcal to about 40 mg/100 kcal of ARA.

The composition may comprise both DHA and ARA. The weight ratio of ARA:DHA may be between about 1:3 and about 9:1. Preferably, the ratio of ARA:DHA may be from about 1:2 to about 4:1. The composition may comprise oils containing DHA and/or ARA. If utilised, the source of DHA and/or ARA may be any source known in the art such as marine oil, fish oil, single cell oil, egg yolk lipid, or brain lipid. The DHA and ARA may be sourced from single cell Martek oils, DHASCO® and ARASCO®, or variations thereof. The DHA and ARA may be in a natural form, provided that the remainder of the LCPUFA source does not result in any substantial deleterious effect on the infant. Alternatively, the DHA and ARA may be used in refined form.

The composition may comprise at least one protein source, wherein the protein source provides protein to the composition. The protein source may comprise intact protein, partially hydrolysed protein, extensively hydrolysed protein, small amino acid peptides, or any combination thereof. The protein source may be present in the composition in addition to another protein source, such as lactoferrin. The protein source may be any used in the art, such as non-fat milk, whey protein, casein, soy protein, hydrolysed protein, amino acids, and the like. Bovine milk protein sources may comprise, but are not limited to, milk protein powders, milk protein concentrates, milk protein isolates, non-fat milk solids, non-fat milk, non-fat dry milk, whey protein, whey protein isolates, whey protein concentrates, sweet whey, acid whey, casein, acid casein, caseinate (e.g. sodium caseinate, sodium calcium caseinate, calcium caseinate) or any combination thereof.

As noted above, the protein source of the composition may comprise partially hydrolysed protein, extensively hydrolysed protein, or a combination thereof. The hydrolysed proteins may be treated with enzymes to break down some or most of the proteins that cause adverse symptoms with the goal of reducing allergic reactions, intolerance, and sensitisation. The proteins may be hydrolysed by any method known in the art.

The composition may comprise between about 1 g and about 7 g of a protein source per 100 kcal. Preferably, the composition may comprise between about 3.5 g and about 4.5 g of a protein source per 100 kcal. The protein source may comprise from about 40% to about 85% whey protein and from about 15% to about 60% casein.

The composition may be substantially free of protein and may comprise free amino acids as a protein equivalent source. The amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and any combination thereof. The amino acids may be branched chain amino acids. The amount of free amino acids in the composition may vary from about 1 to about 5 g/100 kcal. 100% of the free amino acids have a molecular weight of less than 500 Daltons. As the composition may be substantially free of protein and thus, devoid of the proteins that cause adverse symptoms with the goal of reducing allergic reactions, intolerance, and sensitisation in subjects, the composition may be hypoallergenic.

The composition may comprise at least one starch or starch component. The compositions may comprise at least one source of pectin. The composition may comprise a gelatinised and/or pre-gelatinised starch together with pectin and/or gelatinised pectin. The composition may comprise at least one acidic polysaccharide, such as negatively charged pectin. The composition may comprise at least one pectin-derived acidic oligosaccharide (pAOS).

The composition may comprise at least one carbohydrate source, wherein the carbohydrate source provides carbohydrate to the composition. The carbohydrate source may be present in the composition in addition to another carbohydrate source, such as PDX and GOS. The carbohydrate source may comprise lactose, glucose, fructose, maltodextrins, sucrose, starch, maltodextrin, maltose, fructooligosaccharides, corn syrup, high fructose corn syrup, dextrose, corn syrup solids, rice syrup solids, or any combination thereof. Moreover, hydrolysed, partially hydrolysed, and/or extensively hydrolysed carbohydrates may be desirable for inclusion in the composition due to their easy digestibility. More specifically, hydrolysed carbohydrates are less likely to contain allergenic epitopes. The composition may therefore comprise a carbohydrate source comprising hydrolysed or intact, naturally or chemically modified, starches sourced from corn, tapioca, rice, or potato, in waxy or non-waxy forms, such as hydrolysed corn starch.

The amount of the carbohydrate source in the composition may be between about 5 g and about 25 g/100 kcal. Preferably, the amount of carbohydrate source may be between about 6 g and about 22 g/100 kcal. More preferably, the amount of carbohydrate source may be between about 12 g and about 14 g/100 kcal.

The composition may comprise sialic acid. Mammalian brain tissue contains the highest levels of sialic acid because SA is incorporated into brain-specific proteins, such as the neural cell adhesion molecule (NCAM) and lipids (e.g. gangliosides). Sialic acid is therefore believed to play an important role in neural development and function, learning, cognition, and memory.

The composition may comprise sialic acid provided by an inherent source (such as eWPC), exogenous sialic acid, sialic acid from sources (such as cGMP), or any combination thereof. The composition may comprise sialic acid at a level of about 100 mg/L to about 800 mg/L. Preferably, sialic acid is present at a level of about 120 mg/L to about 600 mg/L. More preferably, sialic acid is present at a level of about 140 mg/L to about 500 mg/L. Alternatively, sialic acid may be present in an amount from about 1 mg/100 kcal to about 120 mg/100 kcal. Preferably, sialic acid may be present in an amount from about 14 mg/100 kcal to about 90 mg/100 kcal. More preferably, sialic acid may be present in an amount from about 15 mg/100 kcal to about 75 mg/100 kcal.

The composition may comprise a source of β-glucan. The source of β-glucan may comprise β-1,3-glucan. Preferably, the β-1,3-glucan may be β-1,3;1,6-glucan. The amount of β-glucan present in the composition may be at between about 0.010 and about 0.080 g per 100 g of composition. The amount of β-glucan in the composition may be between about 3 mg and about 17 mg per 100 kcal. Preferably, the amount of β-glucan may be between about 6 mg and about 17 mg per 100 kcal.

The composition may comprise at least one fat or lipid source, wherein the fat or lipid source provides fat and/or lipid to the composition. Suitable fat or lipid sources for the composition may be any known or used in the art. The fat or lipid source may be present in the composition in addition to another fat or lipid source, such as a LCPUFA. The fat or lipid source may comprise animal sources, such as milk fat, butter, butter fat, or egg yolk lipid; marine sources, such as fish oils, marine oils, or single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, or wheat germ oil; medium chain triglyceride oils; emulsions and esters of fatty acids; or any combination thereof.

The composition may comprise between about 1 g/100 kcal and about 10 g/100 kcal of a fat or lipid source. Preferably, the composition may comprise between about 2 g/100 kcal and about 7 g/100 kcal of a fat or lipid source. More preferably, the composition may comprise between about 2.5 g/100 kcal and about 6 g/100 kcal of a fat or lipid source. Most preferably, the composition may comprise between about 3 g/100 kcal and about 4 g/100 kcal of a fat or lipid source.

The composition may comprise choline. Choline is a nutrient that is essential for normal function of cells. Choline is a precursor for membrane phospholipids and it accelerates the synthesis and release of acetylcholine, a neurotransmitter involved in memory storage. Without wishing to be bound by theory, it is believed that dietary choline and docosahexaenoic acid (DHA) act synergistically to promote the biosynthesis of phosphatidylcholine and thus, help promote synaptogenesis in human subjects. Additionally, choline and DHA act synergistically to promote dendritic spine formation, which is important in the maintenance of established synaptic connections. The composition may comprise about 20 mg to about 100 mg of choline per 8 fl. oz. (236.6 mL) serving.

The composition may comprise inositol. The composition may comprise between about 10 mg/100 kcal and 40 mg/100 kcal. Alternatively, the composition may comprise between about 200 mg/L and about 300 mg/L.

The composition may comprise one or more emulsifier, as an emulsifier can increase the stability of the composition. The emulsifier may comprise, but is not limited to, egg lecithin, soy lecithin, alpha lactalbumin, monoglycerides, diglycerides, or any combination thereof. The composition may comprise from about 0.5 wt % to about 1 wt % of emulsifier, based on the total dry weight of the composition. Preferably, the composition may comprise from about 0.7 wt % to about 1 wt % of emulsifier based on the total dry weight of the composition.

The composition may comprise one or more preservative, as a preservative can extend the shelf-life of the composition. The preservative may comprise, but is not limited to, potassium sorbate, sodium sorbate, potassium benzoate, sodium benzoate, calcium disodium EDTA, or any combination thereof. The composition may comprise from about 0.1 wt % to about 1.0 wt % of a preservative based on the total dry weight of the composition. Preferably, the composition may comprise from about 0.4 wt % to about 0.7 wt % of a preservative, based on the total dry weight of the composition.

The composition may comprise one or more stabiliser, as a stabiliser can help preserve the structure of the composition. The stabiliser may comprise, but is not limited to, gum arabic, gum ghatti, gum karaya, gum tragacanth, agar, furcellaran, guar gum, gellan gum, locust bean gum, pectin, low methoxyl pectin, gelatine, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, or any combination thereof.

The composition may be provided in any form known in the art. The composition may take the form of a powder, a gel, a suspension, a paste, a solid, a liquid, a liquid concentrate, a reconstitutable powdered milk substitute, or a ready-to-use product. Preferably, the composition may take the form of a powder, a liquid concentrate, or a ready-to-use product. More preferably, the composition may be provided in a powder form. When the composition is provided in a powder form, the powder may have a particle size in the range of 5 μm to 1500 μm. When the composition is provided in a powder form, the particle size is preferably in the range of 10 μm to 300 μm.

The composition may be intended for a paediatric subject or an adult. The paediatric subject may be an infant or a child. The infant may be a vaginally-delivered infant. Alternatively, the infant may be an infant delivered by C-section. The gut microbiota play a significant role in the development and maturation of the immune system. It is known that the gut microbiota of C-section infants is different to infants that were vaginally delivered, with a study showing that C-section birth is associated with an increased likelihood of immune and metabolic disorders such as allergies, asthma, hypertension, and obesity (Hansen et al., J Immunol Aug. 1, 2014, 193 (3) 1213-1222). One possible way of reducing the likelihood of immune and metabolic disorders in C-section infants may be the provision of a composition comprising beneficial probiotics such as LGG and B. infantis, in an attempt to bring the gut microbiota of the C-section infants into closer alignment with the gut microbiota of vaginally-delivered infants. The composition of the present application, where the viability of beneficial probiotics such as LGG and B. infantis is increased due to the presence of MFGM, may therefore be particularly advantageous for C-section infants.

The composition may comprise a nutritional supplement, an adult's nutritional product, a children's nutritional product, an infant formula, a human milk fortifier, a toddler milk, or any other composition designed for an infant or a paediatric subject. The composition may be provided in an orally-ingestible form, wherein the orally-ingestible comprises a food, a beverage, a tablet, a capsule, or a powder.

The composition may be expelled directly into a subject's intestinal tract. The composition may be expelled directly into the gut. The composition may be formulated to be consumed or administered enterally under the supervision of a physician.

The composition may be delivered to an infant from birth until a time that matches full-term gestation. Alternatively, the composition may be delivered to an infant from birth until at least about three months corrected age, until at least about six months corrected age, or until at least about one-year corrected age. In another aspect, the composition may be delivered to a subject as long as is necessary to correct nutritional deficiencies.

The composition may be suitable for a number of dietary requirements. The composition may be kosher. The composition may be a non-genetically modified product. The composition may be sucrose-free. The composition may also be lactose-free. The composition may not contain any medium-chain triglyceride oil. No carrageenan may be present in the composition. The composition may be free of all gums.

The scope of the present invention is defined in the appended claims. It is to be understood that the skilled person may make amendments to the scope of the claims without departing from the scope of the present disclosure.

### DESCRIPTION OF FIGURES

FIG. 1: (a) Plot of LGG numbers vs incubation time for different source of MFGM concentrations; (b) Bar chart showing LGG numbers after 24 hours for the different source of MFGM concentrations.

FIG. 2: (a) Bar chart showing LGG numbers after 30 minutes in the presence of bile, at different source of MFGM concentrations; (b) Bar chart showing LGG numbers after three hours in the presence of bile, at different source of MFGM concentrations.

FIG. 3: (a) Transmission electron microscopy (TEM) images: (i) TEM image of LGG in the presence of bile alone; (ii) TEM image of LGG in the presence of bile and a source of MFGM; (iii) TEM image of LGG in the presence of a source of MFGM alone; (b) Bar chart showing cell-bound exopolysaccharide production by LGG under different conditions.

FIG. 4: (a) Optical microscopy (OM) images: (i) OM image of LGG in the presence of MRS alone; (ii) OM image of LGG in the presence of bile alone; (iii) OM image of LGG in the presence of bile and a source of MFGM; (iv) OM image of LGG in the presence of a source of MFGM alone; (b) Bar chart showing EPS concentration under different conditions.

FIG. 5: Bar chart for determining LGG biofilm formation under different conditions.

FIG. 6: Bar chart showing the effects of whey powder concentrate (WPC) and a source of MFGM on the bile resistance of LGG.

FIG. 7: (a) Plot showing the amount of LGG in mouse cecum samples 24 hours after the last gavage of MRS, a source of MFGM, LGG alone, or LGG in combination with a source of MFGM; (b) Plot showing the amount of LGG in mouse faecal samples 24 hours after the last gavage of MRS, a source of MFGM, or LGG alone or LGG in combination with a source of MFGM.

### EXPERIMENTAL PROCEDURE

### Study 1: Effect of MFGM on the Growth Profile of LGG

In order to estimate the effects of MFGM on the LGG growth profile, either a broth containing 5 g/L of a source of MFGM (Lacprodan® MFGM-10, Arla Foods Ingredients), or a broth without MFGM (i.e. the control), was inoculated with 1% of resuscitated cells in De Man, Rogosa and Sharpe (MRS) broth. After incubation at 37° C. for 24 hours, cells were diluted and enumerated on MRS agar by the ‘drop plate’ method (Chen, Nace & Irwin, 2003).

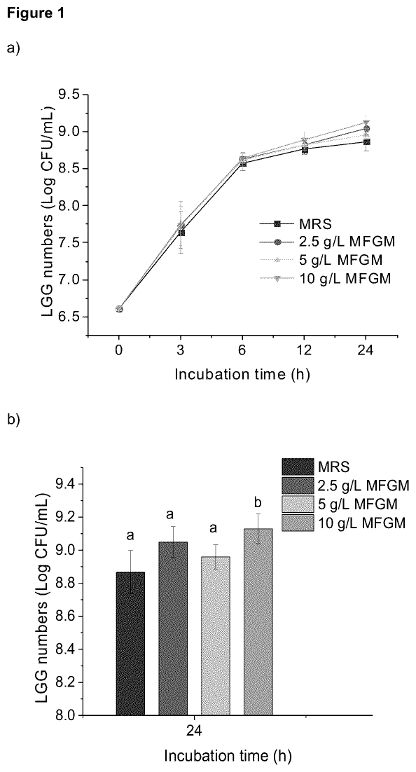

As can be seen in FIG. 1, the presence of MFGM did not change the profile of the LGG growth curve.

### Study 2: Effect of MFGM on the Growth of ‘Infant-Specific Beneficial Bacteria’

In order to estimate the effects of MFGM on the growth of several probiotics, either broth containing 5 g/L of a source of MFGM (Lacprodan® MFGM-10, Arla Foods Ingredients), or a broth without MFGM (i.e. the control), was inoculated with 1% of resuscitated cells in MRS broth (lactobacilli and bifidobacteria), M17 broth (enterococci), or reinforced clostridia medium (RCM; clostridia, bacteroides, cronobacter, staphylococcus). After incubation at 37° C. for 24 hours, cells were diluted and enumerated on MRS agar by the ‘drop plate’ method (Chen, Nace, & Irwin, 2003).

TABLE 2

| | | Inoculated | | |
|---|---|---|---|---|
| | | amount | MRS | MRS + MFGM |
| Bacteria | Medium | (log CFU/mL) | (log CFU/mL) | (log CFU/mL) |
| L. rhamnosus GG | MRS | 6.60 ± 0.04 | 8.86 ± 0.13 | 9.13 ± 0.09 |
| (ATCC 53103) | | | | |
| B. longum subsp. | MRS with 0.05% | 6.91 ± 0.06 | 9.32 ± 0.09 | 9.26 ± 0.06 |
| infantis | cysteine | | | |
| (ATCC 15697) | | | | |
| B. breve | MRS with 0.05% | 7.45 ± 0.02 | 9.48 ± 0.05 | 9.59 ± 0.02 |
| (ATCC 15700) | cysteine | | | |
| B. animalis subsp. | MRS with 0.05% | 7.16 ± 0.06 | 9.12 ± 0.10 | 9.23 ± 0.09 |
| Lactis Bf-6 | cysteine | | | |
| B. infantis | MRS with 0.05% | 7.41 ± 0.03 | 9.43 ± 0.07 | 9.39 ± 0.06 |
| (Commercial isolate) | cysteine | | | |
| B. adolescentis | MRS with 0.05% | 7.06 ± 0.20 | 8.39 ± 0.47 | 8.64 ± 0.43 |
| (ATCC 15703) | cysteine | | | |
| B. bifidum | M17 with 2% | 6.72 ± 0.18 | 8.99 ± 0.00 | 8.92 ± 0.14 |
| (ATCC 29521) | glucose | | | |

The results detailed in Table 2 show that supplements of MFGM into media containing different bacteria did not significantly change the growth of the probiotics after 24 hours of incubation.

### Study 3: Effect of MFGM on the Bile Resistance of LGG

To determine the effect of MFGM on the bile resistance of LGG, LGG cells were grown overnight in MRS, then centrifuged, washed with autoclaved water, and resuspended in an equal volume of different kinds of MRS broth. Five different LGG samples were then prepared: (i) MRS only; (ii) MRS+0.5% porcine bile extract (Sigma, MI, USA; weight per volume (w/v)); (iii) to (v) MRS+0.5% porcine bile extract+a source of MFGM (Lacprodan® MFGM-10, Arla Foods Ingredients) at a concentration of 2.5 g/L, 5 g/L, and 10 g/L, respectively. The tubes were anaerobically incubated at 37° C. for 30 minutes or three hours. The cells were sampled, diluted in phosphate-buffered saline (PBS), and enumerated on MRS agar.

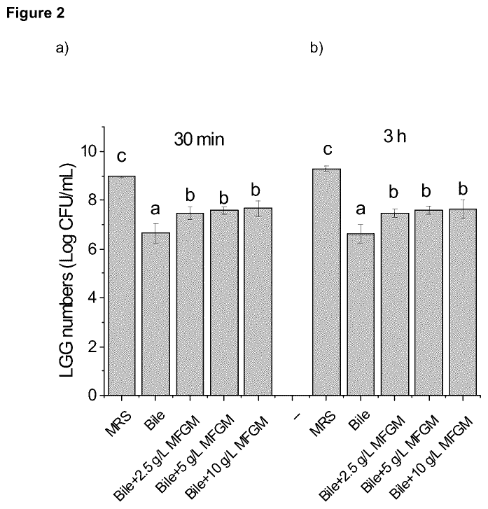

As can be seen in FIG. 2, the presence of MFGM increases the survival of LGG in the presence of bile. More specifically, the presence of 0.5% porcine bile caused a 2.3 log CFU/mL reduction in LGG numbers. In the presence of MFGM, the presence of 0.5% porcine bile only reduced LGG numbers by 1.3 to 1.5 log CFU/mL. Therefore, the presence of MFGM resulted in a 1 log CFU/mL increase in LGG numbers, compared with the LGG numbers in the absence of MFGM. Although the bile-protecting effect of MFGM was only investigated for LGG in this study, there is nothing in the literature to suggest, and no reason to believe, that MFGM would not exhibit such a bile-protecting effect for many other probiotics, such as B. infantis, Bifidobacterium animalis subsp. lactis, etc.

### Study 4: Effect of Different Batches of MFGM on the Bile Resistance of LGG

To determine if different batches and/or suppliers of a source of MFGM vary in their bile-protecting effects, LGG cells were grown overnight in MRS and washed once with autoclaved water. The LGG cells were then resuspended in the same volume of MRS with bile and/or different batches of a source of MFGM, as indicated. After 30 minutes of anaerobic incubation at 37° C., the cells were diluted and enumerated on MRS agar, by drop plate method.

TABLE 3

| | LGG numbers |
|---|---|
| Treatment | (log CFU) |
| MRS | 9.32 ± 0.03 |
| MRS + 5 g/L of Lacprodan ® MFGM-10 | 9.34 ± 0.02 |
| (Arla Foods Ingredients) | |
| MRS + 0.5% Bile | 5.70 ± 0.43 |
| MRS + 0.5% Bile + 5 g/L Lacprodan ® MFGM-10 #1 | 6.66 ± 0.67 |
| MRS + 0.5% Bile + 5 g/L Lacprodan ® MFGM-10 #4 | 6.00 ± 0.42 |
| MRS + 0.5% Bile + 5 g/L Lacprodan ® MFGM-10 #5 | 6.35 ± 0.75 |
| MRS + 0.5% Bile + 5 g/L Lacprodan@ MFGM-10 #6 | 6.54 ± 0.63 |
| MRS + 0.5% Bile + 5 g/L Hilmar ™ 7500 MFGM | 6.46 ± 0.28 |
| (Hilmar Ingredients) #7 | |
| MRS + 0.5% Bile + 5 g/L Hilmar ™ 7500 MFGM #9 | 6.27 ± 0.39 |

As shown in Table 3, there is no significant difference in the bile-protecting effects of different batches of MFGM.

### Study 5: Elucidation of the Mechanism of MFGM Action

### Study 5a: Transmission Electron Microscopy (TEM) Analysis

When LGG cells are stressed by the presence of bile, they produce exopolysaccharide (EPS) to form a capsular polysaccharide (CPS), in an attempt to protect themselves from the bile. The presence and nature of the interactions of the CPS and MFGM with LGG was investigated by TEM.

LGG cells were grown in MRS with or without a source of MFGM (Lacprodan®MFGM-10, Arla Foods Ingredients), and/or with or without 0.5% bile. The LGG cells were then washed once with PBS and suspended in Karnovsky's fixative containing 2% glutaraldehyde and 2.5% paraformaldehyde. Then, the samples were stained with 2% uranyl acetate for one minute and observed by a JEOL 2100 cryo-TEM. CPS was extracted from the bacterial cells according to the Tallon et al. method (Tallon, Bressollier & Urdaci, 2003). The CPS concentrations in the different culture media were determined using the ‘phenol-sulphuric acid method’ (Masuko, et al. 2005), using glucose as a standard. The results were expressed in μg of glucose per 10¹⁰ CFU.

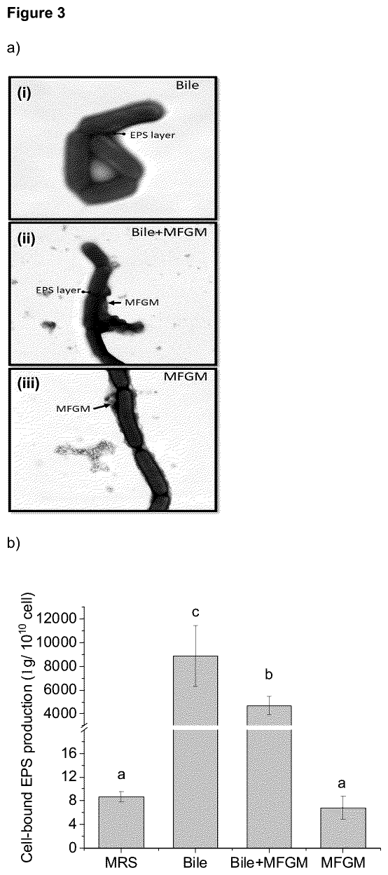

TEM analysis shows that a CPS layer was nearly absent in LGG grown in the MRS broth without bile, regardless of whether or not MFGM was present (see FIG. 3(a)(i)). The measurement of EPS production also shows that little EPS was produced in the MRS broth in the absence of bile, regardless of the supplementation of MFGM (see FIG. 3(b)). However, when stressed by the bile, the LGG cells were surrounded by a CPS layer. The CPS layer was much thinner under bile stress in the presence of MFGM, than without MFGM (see FIGS. 3(a)(ii) and 3(a)(iii)). The TEM images also show that MFGM interacts with the LGG cells (see FIGS. 3(a)(ii) and 3(a)(iii)). The exact nature of the interaction between LGG and MFGM has not yet been definitively established but it is possible that there seems to be a form of ‘encapsulation’ of the LGG by MFGM. Further, when the LGG cells were stressed by the presence of bile, MFGM also reduced the EPS production of LGG significantly (4,683±797 μg/10¹⁰ CFU; p<0.05) compared to LGG in the presence of bile without MFGM (8,872±2587 μg/10¹⁰ CFU; see FIG. 3b); this is a 48% reduction in EPS production due to the presence of MFGM.

Although the ‘encapsulating effect’ of MFGM has only been investigated for LGG in this study, there is nothing in the literature to suggest, and no reason to believe, that MFGM would not exhibit such an ‘encapsulating effect’ for many other probiotics, such as B. infantis, Bifidobacterium animalis subsp. lactis, etc.

In summary, when MFGM is present during the bile-induced stress of LGG cells, the LGG cells make less EPS. This supports our earlier results that MFGM protects LGG cells from bile stress.

### Study 5b: Optical Microscopy Analysis

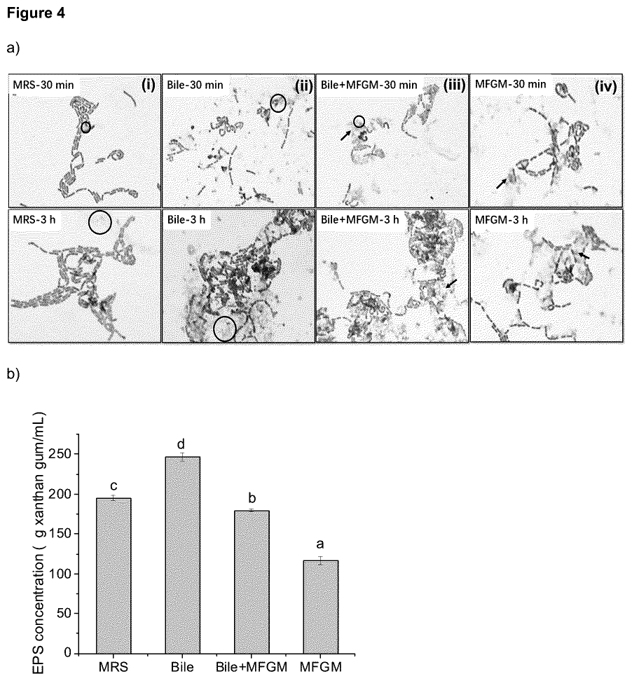

Four LGG cell samples were prepared: (i) MRS only; (ii) MRS+0.5% porcine bile; (iii) MRS+0.5% porcine bile+a source of MFGM (Lacprodan® MFGM-10, Arla Foods Ingredients); and, (iv) MRS+a source of MFGM (Lacprodan® MFGM-10, Arla Foods Ingredients). The samples were stained by Alcian blue and safranin solution, and observed by optical microscopy after 30 minutes and three hours. When 0.5% bile was added to the MRS broth, the amount of EPS produced by the LGG cells increased dramatically (see FIG. 4a). Moreover, LGG cells, in the absence of MFGM, were shown to be surrounded by an EPS matrix after three hours. However, when LGG cells were stressed by bile in the presence of MFGM, the amount of EPS around the LGG cells was dramatically reduced. The EPS was purified and the amount of EPS was quantitatively determined by Alcian blue stain method (see FIG. 4b).

As FIGS. 4a and 4b show, the presence of bile increased the EPS production of LGG cells, but the addition of MFGM decreased the amount of EPS produced by the LGG cells in the presence of bile.

### Study 5c: Measurement of Biofilm Formation

The measurement of biofilm formation by LGG in different media was performed in a 96-well plate as previously described by Lebeer et al. (Applied and Environmental Microbiology, 73, 6768-6775). Briefly, an overnight LGG culture was centrifuged, washed with water, and then resuspended into MRS, MRS+bile, MRS+a source of MFGM (Lacprodan® MFGM-10, Arla Foods Ingredients), and MRS+bile+a source of MFGM (Lacprodan® MFGM-10, Arla Foods Ingredients). For biofilm formation, a 96-well plate was loaded with 200 μL of media and anaerobically incubated, without shaking, for 72 hours at 37° C. Four replicates for each strain were used for each assay and three biologically-independent experiments were conducted. To measure biofilm formation, the 96-well plate was washed in water and the attached LGG were stained with 200 μL of 0.1% (w/v) crystal violet for 30 minutes. Unbound stain was washed off with water. After air drying for 30 minutes, the cell bound crystal violet was dissolved in 200 μL of ethanol/acetone (80:20) solution for 10 minutes. The absorbance (A595) of 135 μL of each well was measured with a Multiscan Accent machine (Thermo Fisher, USA).

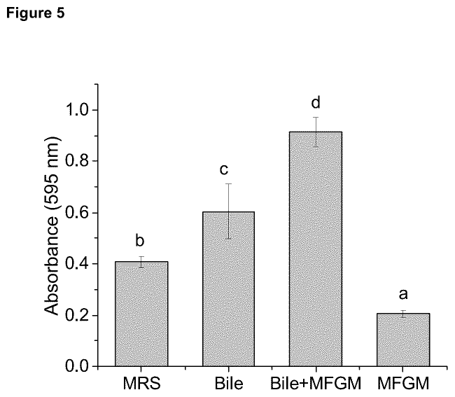

As shown in FIG. 5, LGG biofilm formation was enhanced by the presence of bile, and was further enhanced when the LGG cells were exposed to bile in the presence of MFGM. The observation that LGG biofilm formation was increased in the presence of bile and MFGM, in comparison to bile only, once again suggests that MFGM is able to increase the viability of LGG cells in the presence of bile.

### SUMMARY

The results show that LGG produces less EPS in the presence of bile and MFGM, than in the presence of bile alone. The TEM images suggest that MFGM interacts with the LGG in such a manner that it forms a type of protective layer for the LGG cells. Finally, MFGM was found to enhance the biofilm formation of LGG.

### Study 6: Effect of MFGM on the Bile Resistance of Other Bacteria

The particular probiotic cells were grown overnight in MRS and washed once with autoclaved water. 1% of the particular probiotic cells was then resuspended in the same volume of MRS with 0.5% porcine bile and/or 5 g/L of a source of MFGM (Lacprodan® MFGM-10, Arla Foods Ingredients). After 30 minutes, and also three hours, of anaerobic incubation at 37° C., the cells were diluted and enumerated on MRS agar by drop plate method.

### Bile-Sensitive Probiotics

TABLE 4

| | | B. infantis | B. longum | L. rhamnosus |
|---|---|---|---|---|
| | | (Commercial | subsp. infantis | GG |
| Treatment | | isolate) | (ATCC 15697) | (ATCC 53103) |
| 30 | MRS | 8.93 ± 0.01 | 8.93 ± 0.01 | 8.98 ± 0.03 |
| min | MFGM | 8.94 ± 0.07 | 8.94 ± 0.07 | 8.99 ± 0.03 |
| | Bile | 3.57 ± 0.99 | 3.57 ± 0.99 | 6.65 ± 0.40 |
| | Bile + MFGM | 7.41 ± 0.59 | 7.41 ± 0.59 | 7.57 ± 0.15 |
| 3 | MRS | 9.20 ± 0.15 | 9.20 ± 0.15 | 9.32 ± 0.03 |
| hrs | MFGM | 9.12 ± 0.17 | 9.12 ± 0.17 | 9.33 ± 0.02 |
| | Bile | 3.00 ± 0.00 | 3.00 ± 0.00 | 6.63 ± 0.38 |
| | Bile + MFGM | 3.25 ± 0.25 | 3.25 ± 0.25 | 7.60 ± 0.16 |

As can be seen in Table 4, the presence of MFGM increases the numbers of bile-sensitive probiotics, after three hours, compared to the numbers of the probiotics in the absence of MFGM.

### Intermediate Bile-Sensitive Probiotics

TABLE 5

| | | B. | L. | | L. |
|---|---|---|---|---|---|
| | | adolescentis | reuteri | L. | gasseri |
| | | (ATCC | (DSM | acidophilus | (ATCC |
| Treatment | | 15703) | 17938) | NCFM | 33323) |
| 30 | MRS | 8.92 ± 0.41 | 9.50 ± 0.04 | 8.87 ± 0.09 | 9.17 ± 0.13 |
| min | MFGM | 8.94 ± 0.43 | 9.46 ± 0.04 | 8.91 ± 0.22 | 8.98 ± 0.17 |
| | Bile | 8.40 ± 0.68 | 9.06 ± 0.04 | 8.31 ± 0.02 | 8.91 ± 0.15 |
| | Bile + | 8.76 ± 0.39 | 9.16 ± 0.18 | 8.48 ± 0.12 | 8.99 ± 0.15 |
| | MFGM | | | | |
| 3 | MRS | 9.03 ± 0.25 | 9.40 ± 0.14 | 8.90 ± 0.19 | 9.25 ± 0.04 |
| hrs | MFGM | 9.09 ± 0.24 | 9.59 ± 0.07 | 9.06 ± 0.24 | 9.30 ± 0.08 |
| | Bile | 5.99 ± 0.26 | 7.41 ± 0.55 | 8.01 ± 0.12 | 8.42 ± 0.31 |
| | Bile + | 6.33 ± 0.23 | 8.73 ± 0.36 | 8.46 ± 0.00 | 8.88 ± 0.11 |
| | MFGM | | | | |

As can be seen in Table 5, the presence of MFGM increases the numbers of intermediate bile-sensitive probiotics, after three hours, compared to the numbers of the probiotics in the absence of MFGM.

### Study 7: Comparison of the Effect of Whey Powder Concentrate (WPC) and MFGM on the Bile Resistance of LGG

LGG cells were grown overnight in MRS, then centrifuged, washed with autoclaved water, and resuspended in an equal volume of different kinds of MRS broth. 12 LGG cell samples were then prepared in tubes: (i) MRS only; (ii) MRS+WPC1*; (iii) MRS+WPC2*; (iv) MRS+WPC3*; (v) MRS+a source of MFGM* (Lacprodan® MFGM-10, Arla Foods Ingredients); (vi) MRS+a source of MFGM Lacprodan® MFGM-10, Arla Foods Ingredients); (vii) MRS+0.5% porcine bile; (viii) MRS+0.5% porcine bile+WPC1*; (ix) MRS+0.5% porcine bile+WPC2*; (x) MRS+0.5% porcine bile+WPC3*; (xi) MRS+0.5% porcine bile+a source of MFGM*; and, (xii) MRS+0.5% porcine bile+a source of MFGM, wherein: ‘WPC1*’, ‘WPC2*’, and ‘WPC3*’ refer to three different WPC batches; ‘a source of MFGM*’ refers to autoclaved WPC enriched with MFGM; and, ‘a source of MFGM’ refers to un-autoclaved WPC enriched with MFGM. The concentration of the WPC or Lacprodan® MFGM-10 in the samples was 5 g/L. The tubes were anaerobically incubated at 37° C. for 30 minutes or three hours. The cells were sampled, diluted in PBS, and enumerated on MRS agar.

The results are from three independent experiments. The statistical tests were performed on three independent repetitions and analysed using a one-way ANOVA and a Tukey's test. The results were expressed as a mean value, with the standard deviation shown. Different superscript letters were used to indicate a significant difference when p<0.05.

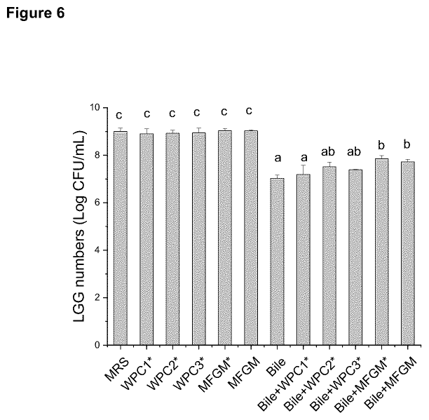

As FIG. 6 shows, 0.5% porcine bile caused a significant reduction in LGG numbers after 30 minutes incubation. The results show that MFGM improves the bile resistance of LGG in a much more pronounced manner than WPC. This confirms that the LGG-protecting effect exhibited is specific for MFGM and not caused by other WPC components.

### Study 8: Mouse Model of the Effect of MFGM on LGG Survival

Healthy, male six-week-old BALB/c mice (n=32; body weight=20 to 26 g) were gavaged for three days, with one of the following treatments: 0.1 mL of MRSC; MRSC with a source of MFGM (Lacprodan® MFGM-10, Arla Foods Ingredients; 5 g/L); MRSC with LGG (5×10⁷ CFU/mL); or, MRSC with a source of MFGM (Lacprodan® MFGM-10, Arla Foods Ingredients; 5 g/L) and LGG (5×10⁷ CFU/mL). On day three, faecal samples were collected. During faecal collection, the mice were moved to a new cage 24 hours after the last gavage. After one hour in the cage, all faecal pellets in this new cage were then collected with tweezers and stored at −20° C. for eventual microbial DNA extraction. After faecal collection, the mice from each group were euthanised by cervical dislocation under anaesthesia by CO₂. The cecum contents were then collected and stored at −20° C., for microbial DNA extraction. Total DNA was extracted from caecal contents and faeces using E.Z.N.A.® Stool DNA Kit. LGG specific primers were used to detect LGG. Each sample was amplified by qPCR in triplicate. The statistical tests were performed on three independent repetitions and analysed using a one-way ANOVA and a Tukey's test. Results were expressed as means of three independent experiments±S.D. (Standard Deviation). Differences were considered statistically significant when p<0.05.

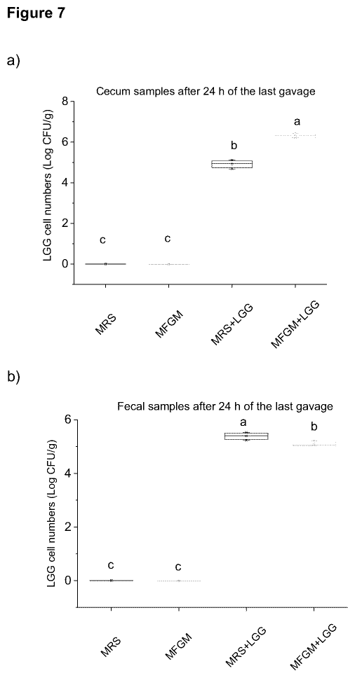

As shown in FIG. 7a), the combination of LGG and MFGM resulted in a 25.5-fold increase in LGG concentration in mice caecum samples, when compared to the group receiving LGG only. FIG. 7b) shows that the faecal LGG concentration in mice gavaged with LGG only was higher (2.54×10⁵ CFU/g), than the faecal LGG concentration when the mice were gavaged with a combination of LGG and MFGM (1.3×10⁵ CFU/g). The results illustrate how the presence of LGG in combination with MFGM results in a greater tendency for the LGG to remain in the mice GI system, and avoid excretions, when compared to LGG alone.

In summary, the LGG-protecting effect exhibited by MFGM in the previous in vitro studies has been corroborated in vivo. Although the bile-protecting effect of MFGM was only investigated for LGG in the in vivo experiment, there is nothing in the literature to suggest, and no reason to believe, that MFGM would not exhibit such a bile-protecting effect for many other probiotics, such as B. infantis, Bifidobacterium animalis subsp. lactis, etc.

### Example Compositions

The compositions shown in Table 6 illustrate examples of compositions within the scope of the present disclosure, but are in no way intended to provide any limitation on the disclosure.

TABLE 6

| | Composition | | | | | | | | | | |
|---|---|---|---|---|---|---|---|---|---|---|---|
| Component | I | II | III | IV | V | VI | VII | VIII | IX | X | XI |
| MFGM (derived | ✓ | ✓ | ✓ | ✓ | ✓ | ✓ | ✓ | ✓ | ✓ | ✓ | ✓ |
| from eWPC) | | | | | | | | | | | |
| LGG | X | ✓ | ✓ | ✓ | ✓ | ✓ | ✓ | ✓ | X | X | ✓ |
| B. infantis | X | X | X | X | X | X | X | X | ✓ | ✓ | ✓ |
| PDX | X | X | ✓ | X | X | X | ✓ | ✓ | X | ✓ | ✓ |
| GOS | X | X | ✓ | X | X | X | ✓ | ✓ | X | ✓ | ✓ |
| DHA | X | X | X | ✓ | ✓ | X | ✓ | ✓ | X | ✓ | ✓ |
| ARA | X | X | X | X | ✓ | X | ✓ | ✓ | X | ✓ | ✓ |
| Lactoferrin | X | X | X | X | X | ✓ | X | ✓ | X | ✓ | ✓ |

| | Composition | | | | | | | | | | |
|---|---|---|---|---|---|---|---|---|---|---|---|
| Component | XII | XIII | XIV | XV | XVI | XVII | XVIII | XIX | XX | XXI | XXII |
| MFGM | ✓ | ✓ | ✓ | ✓ | ✓ | ✓ | ✓ | ✓ | ✓ | ✓ | ✓ |
| (derived from | | | | | | | | | | | |
| buttermilk) | | | | | | | | | | | |
| LGG | X | ✓ | ✓ | ✓ | ✓ | ✓ | ✓ | ✓ | X | X | ✓ |
| B. infantis | X | X | X | X | X | X | X | X | ✓ | ✓ | ✓ |
| PDX | X | X | ✓ | X | X | X | ✓ | ✓ | X | ✓ | ✓ |
| GOS | X | X | ✓ | X | X | X | ✓ | ✓ | X | ✓ | ✓ |
| DHA | X | X | X | ✓ | ✓ | X | ✓ | ✓ | X | ✓ | ✓ |
| ARA | X | X | X | X | ✓ | X | ✓ | ✓ | X | ✓ | ✓ |
| Lactoferrin | X | X | X | X | X | ✓ | X | ✓ | X | ✓ | ✓ |

Key:
✓ = present;
X = not present

1-19. (canceled)

20. A method for preserving viability of a probiotic comprising providing a composition comprising a probiotic and milk fat globule membrane.

21. The method of claim 20, wherein preserving viability of a probiotic comprises preserving, or limiting the reduction in, viability of the probiotic in the presence of bile.

22-25. (canceled)

26. The method of claim 20, wherein the probiotic comprises a Bifidobacterium species, a Lactobacillus species, or a combination thereof.

27. The method of claim 26, wherein the probiotic comprises Bifidobacterium longum subsp. infantis, Lactobacillus rhamnosus GG (ATCC number 53103), or a combination thereof.

28. The method of claim 27, wherein the probiotic comprises Lactobacillus rhamnosus GG (ATCC number 53103).

29. The method of claim 20, wherein the milk fat globule membrane is provided by an enriched milk product, buttermilk, or a combination thereof.

30. The method of claim 20, wherein the milk fat globule membrane is derived from at least one of bovine milk, bovine cream, porcine milk, equine milk, buffalo milk, goat milk, murine milk, camel milk, or any combination thereof.

31. The method of claim 20, wherein the composition is intended for a paediatric subject.

32. The method of claim 31, wherein the composition is an infant formula, a follow-up formula, or a toddler milk.

33. The method of claim 31, wherein the paediatric subject is an infant.

34. The method of claim 33, wherein the infant is an infant delivered by caesarean section.

35. The method of claim 20, wherein the composition is intended for an adult.

36. The method of claim 20, wherein the composition is a synthetic composition.

37. A method for reducing bile-induced stress of a probiotic comprising providing a composition comprising a probiotic and milk fat globule membrane.

38. The method of claim 37, wherein the probiotic comprises a Bifidobacterium species, a Lactobacillus species, or a combination thereof.

39. The method of claim 38, wherein the probiotic comprises Lactobacillus rhamnosus GG (ATCC number 53103).

40. The method of claim 37, wherein the milk fat globule membrane is provided by an enriched milk product, buttermilk, or a combination thereof.

41. The method of claim 37, wherein the milk fat globule membrane is derived from at least one of bovine milk, bovine cream, porcine milk, equine milk, buffalo milk, goat milk, murine milk, camel milk, or any combination thereof.

42. The method of claim 37, wherein the composition is intended for a paediatric subject.

43. The method of claim 37, wherein the composition is a synthetic composition.
